(19) United States
(12) Patent Application Publication
(10) Pub. No.: US 2017/0307640 A1
(43) Pub. Date: Oct. 26, 2017

(54) METHODS, KITS AND DEVICES FOR DETECTING BII-SPECTRIN, AND BREAKDOWN PRODUCTS THEREOF, AS BIOMARKERS FOR THE DIAGNOSIS OF NEURAL INJURY

(71) Applicant: BANYAN BIOMARKERS, INC., Alachua, FL (US)

(72) Inventors: Firas KOBAISSY, Alachua, FL (US); Joy GUINGAB, Alachua, FL (US); Kevin Ka-Wang WANG, Gainesville, FL (US); Ronald L. HAYES, Alachua, FL (US)

(73) Assignee: BANYAN BIOMARKERS, INC., Alachua, FL (US)

(21) Appl. No.: 15/630,734

(22) Filed: Jun. 22, 2017

Related U.S. Application Data
(63) Continuation of application No. 13/947,599, filed on Jul. 22, 2013.
(60) Provisional application No. 61/673,870, filed on Jul. 20, 2012.

(51) Int. Cl. G01N 33/68

(52) U.S. Cl. CPC G01N 33/6896

(57) ABSTRACT

The present invention identifies biomarkers that are diagnostic of neural injury, neuronal disorder or neurotoxicity and is related to the discovery that proteases are selectively activated in subjects suffering from nervous system damage as compared to healthy subjects. Breakdown products reflecting protease activation are produced and detection of these different biomarkers of the invention is also diagnostic of the degree of severity and type of nerve damage in a subject.

### CROSS REFERENCE TO RELATED APPLICATIONS

This application is a continuation of U.S. patent application Ser. No. 13/947,599 filed Jul. 22, 2013, which claims priority to U.S. Provisional Application No. 61/673,870 filed Jul. 20, 2012. The contents of these applications are incorporated herein by reference in their entireties.

### FIELD OF THE INVENTION

The invention provides for the reliable detection and identification of biomarkers important for the diagnosis and prognosis of nerve cell damage, neural injury, neuronal disorders or neurotoxicity or injury in patients with brain damage. The invention provides for methods, kits and devices for the detection of neural injury, neuronal disorder or neurotoxicity by analyzing the biomarker panel from a patient for specific protein and protein fragments produced in response to the activation of particular proteases. These techniques provide simple yet sensitive approaches to rapidly diagnosing the scope of damage to the brain using biological fluids.

### BACKGROUND

Injury to the brain is a major health concern worldwide and the prognosis for such injury ranges from debilitating to terminal. Treatment efficacy depends upon rapid diagnosis and administration of treatment and as such it is crucial to determine whether there has been insult to the brain and the severity of the resulting injury.

Brain injuries have a wide variety of etiology: traumatic, ischemic, or chemical, and thus may be very difficult to quickly diagnose in an emergency setting. Current technological diagnostics include computed tomography (CT) and magnetic resonance imaging (MRI) scans. Both of these scans are expensive, cumbersome, and not readily deployed in an emergency room setting. These expensive machines often are not available outside of major hospitals and metropolitan areas. Furthermore, CT and MRI scans are not effective for diagnosing mild to moderate brain injuries as these injuries most often do not manifest in physical scans.

To complicate matters, many brain injuries such as those of resulting from chemical etiology have a delayed onset and can avoid initial diagnostic detection by scan entirely. Thus many brain injuries go untreated or misdiagnosed as current diagnostic methods are simply inadequate for diagnosing neural injury, neuronal disorders or neurotoxicity and especially so in mild to moderate cases.

A number of biomarkers have been identified as being associated with neural injuries such as traumatic brain injury (TBI) and neurotoxicity. Understanding how multiple biomarkers overlap and any correlations to injury severity remains unestablished. This lack of understanding is particularly prevalent with respect to neural injuries and disorders.

Biomarkers represent a unique approach to provide objective information and insight in the pathophysiology and the biochemical response of the brain following several types of neural injuries. A number of studies have been conducted on biomarkers in the acute and subacute phase after TBI, but little is known about the role of biochemical markers and their potential use in the later chronic phase after TBI.

Spectrin is a cytoskeletal protein essential for the determination of cell shape, the resilience of membranes to mechanical stress, the positioning of particular transmembrane proteins within the plane of a membrane, and the organization of organelles and molecular traffic. βII-spectrin proteins and their breakdown products (βII-SBDP's) intracellular locations reveal two proteins in muscle cells and neurons. In particular skeletal muscle and heart M line regions contain a short form of the protein; the distal portions of cerebellar granule cell neurites are enriched with a long form of the protein.

Despite the advancements in biomarker technology, no diagnostic or prognostic detection method or device exists for neural injuries or neuronal disorders. Accordingly, a need exists for accessible, inexpensive, simple and specific diagnostic clinical assessments of brain injury and severity so treatment efficacy may be improved. Identification of neurochemical markers that would help determine the existence and severity of brain injury, anatomical and cellular pathology of the damage, and implementation of appropriate medical management and treatment techniques would be particularly useful in improving current medical science.

### SUMMARY OF THE INVENTION

The current invention provides neuronal protein markers that are differentially present in samples of subjects suffering from neural injury as compared to samples of control subjects. The present invention also provides sensitive and rapid methods and kits able to be utilized as diagnostic aids, and in vitro diagnostic devices, for detecting neural injury by detecting these markers. The measurement of these proteins and/or protein fragments produced by specific protease cascades, alone or in combination, in subject samples provides information that a diagnostician can correlate with diagnosis of the existence, type, and severity of neural injury.

In one embodiment, the biomarkers are βII-spectrin and βII-spectrin breakdown products (βII-SBDPs) generated by calpain-2 and/or caspase-3 proteolysis.

In at least one embodiment, at least one biomarker, such as a protein, peptide, variant or fragment thereof, is used to detect a neural injury, neuronal disorder or neurotoxicity in a subject, wherein said at least one biomarker is βII-spectrin, βII-SBDP-80, βII-SBDP-85, βII-SBDP-108, or βII-SBDP-110.

In at least one embodiment, a plurality of biomarkers, such as proteins, peptides, variant or fragment thereof, is used to detect a neural injury, neuronal disorder or neurotoxicity in a subject, where said plurality biomarker is βII-spectrin, βII-SBDP-80, βII-SBDP-85, βII-SBDP-108, βII-SBDP-110 or combinations thereof.

In at least one embodiment, the method for detecting neural injuries, neural disorders or neurotoxicity, include: (a) providing a biological sample isolated from a subject at risk or suspected of having neural injuries, neural disorders or neural toxicity, the sample being a biological fluid in communication with the nervous system of subject; (b) detecting in the sample the presence or amount of at least one marker selected from proteolytic cleavage of βII-spectrin by at least one protease selected from the group consisting of calpain-2 and caspase-3; and (c) correlating the presence or amount of the at least one marker with presence or type of neural injury, neuronal disorder or neurotoxicity in a subject.

In at least one embodiment, the subject will preferably be a human patient suspected of having a damaged nerve cell and the markers being assessed can be one, two, three, four, all, or any combination of βII-spectrin, βII-SBDP-80, βII-SBDP-85, βII-SBDP-108, and βII-SBDP-110.

In at least one embodiment, the biological sample is cerebrospinal fluid (CSF), blood, plasma, serum, saliva, or urine.

In at least one embodiment, the step (b) of detecting in the sample the presence or amount of at least one marker selected from βII-spectrin and/or a βII-SBDPs generated from proteolytic cleavage of βII-spectrin by at least one protease selected from the group consisting of calpain-2 and caspase-3 can include contacting the sample or a portion of the sample with an agent that specifically binds the marker. The agent may be one that does not specifically bind at least one of βII-spectrin, βII-SBDP-80, βII-SBDP-85, βII-SBDP-108, and βII-SBDP-110. (i.e. one that binds only a subset of this group); or one that specifically binds only one of βII-spectrin, βII-SBDP-80, βII-SBDP-85, βII-SBDP-108, and βII-SBDP-110. (i.e. a monospecific agent).

In at least one embodiment, the step (b) of detecting in the sample the presence or amount of at least one marker selected from βII-spectrin and/or a βII-SBDP generated from proteolytic cleavage of βII-spectrin by at least one protease selected from the group consisting of calpain-2 and caspase-3 includes immobilizing the sample or portion thereof on a substrate and/or contacting the substrate with an agent that specifically binds the marker.

In at least one embodiment, the agent contacted with the sample is preferably an antibody.

In at least one embodiment, the step (c) of correlating the presence or amount of the marker with presence or type of cell damage in the subject can include comparing the presence or amount of the marker in the sample with that in a standard sample known to not contain the marker (e.g. a negative control); and/or comparing the presence or amount of the marker in the sample with that in a standard sample known to contain a known amount of the marker (e.g. a positive control).

In at least one embodiment, the invention provides a mixture that includes: (a) a biological sample isolated from a subject suspected of having a neural injury, neural disorder or neurotoxicity, the biological sample being a fluid in communication with the nervous system of the subject prior to being isolated from the subject; and (b) an agent that specifically binds at least one marker selected from of detecting in the sample the presence or amount of at least one marker selected from βII-spectrin and/or a βII-SBDP generated from proteolytic cleavage of βII-spectrin by at least one protease selected from the group consisting of calpain-2 and caspase-3.

In at least one embodiment, the biological sample of the mixture is preferably derived from a human patient suspected of having a damaged nerve cell and the markers being assessed can be one, two, three, four, all, or any combination of βII-SBDP-80, βII-SBDP-85, βII-SBDP-108, and βII-SBDP-110.

In at least one embodiment the agent within the mixture is preferably antibody.

In at least one embodiment, the mixture of the invention can be immobilized on a substrate to facilitate detection by immunoblot or other assay.

In at least one embodiment, the mixture of the invention may further include a detectable label such as one conjugated to the agent, or one conjugated to a substance that specifically binds to the agent (e.g. a detectable secondary agent).

In at least one embodiment, the invention provides a kit for analyzing cell damage that includes: (a) a substrate for holding a biological sample isolated from a subject suspected of having a damaged nerve cell, the biological sample being a fluid in communication with the nervous system of the subject prior to being isolated from the subject; (b) an agent that specifically binds at least one marker selected from βII-spectrin and a βII-SBDP generated from proteolytic cleavage of βII-spectrin by at least one protease selected from the group consisting of calpain-2 and caspase-3; and (c) printed instruction for reacting the agent with the biological sample or portion thereof to detect the presence or amount of the at least one marker in the biological sample.

In at least one embodiment, the sample analyzed in the kit is preferably derived from a human suspected of having a damaged nerve cell and the markers being assessed can be one, two, three, four, all, or any combination of βII-spectrin, βII-SBDP-80, βII-SBDP-85, βII-SBDP-108, and βII-SBDP-110.

In at least one embodiment the agent of the kit can be one that does not specifically bind at least one of βII-spectrin, βII-SBDP-80, βII-SBDP-85, βII-SBDP-108, and βII-SBDP-110; or one that specifically binds only one of βII-spectrin, βII-SBDP-80, βII-SBDP-85, βII-SBDP-108, and βII-SBDP-110.

In at least one embodiment, an in vitro diagnostic device, either colorimetric or electronic, is used which incorporates the use of an ELISA that detects one or more biomarkers of βII-spectrin, βII-SBDP-80, βII-SBDP-85, βII-SBDP-108, βII-SBDP-110, or combinations thereof.

### BRIEF DESCRIPTION OF THE DRAWINGS

FIG. 1 is an immunoblot analysis showing tissue distribution of βII-spectrin protein expression vs. aII-spectrin in rat tissues. βII-spectrin is predominantly expressed in brain tissue as shown with minimal expression in the kidney, lung, and heart tissue.

FIG. 2 is an immunoblot analysis showing βII-SBDPs after 24 hour exposure to various neurotoxic conditions (MTX, STS, EDTA, and NMDA).

FIG. 3 is an immunoblot analysis showing the effects of calpain-2 and caspase-3 inhibitors on degradation patterns of βII-spectrin and all-spectrin in rat cerebrocortical structures.

FIG. 4 is an immunoblot analysis showing βII-SBDP formation in rat cortex of naïve, sham, and TBI groups 48 hours post-CCI.

FIG. 5 is an immunoblot analysis showing βII-SBDP formation in rat hippocampus of naïve, sham, and TBI groups 48 hours post-CCI.

FIG. 6 is an (A) immunoblot analysis and (B) graph showing the temporal profile of βII-SBDP in rat cortex of naïve, sham, and TBI groups at up to 14 days post-CCI.

FIG. 7 is an (A) immunoblot analysis and (B) graph showing the temporal profile of βII-SBDP in rat hippocampus of naïve, sham, and TBI groups at up to 14 days post-CCI.

FIG. 8 is an immunoblot analysis showing the comparison of βII-spectrin protein proteolytic fragmentation after brain cortex digestion with calpain-2 and caspase-3 proteases.

FIG. 9 is a schematic illustration of the putative calpain-2 and caspase-3 cleavage sites in βII-spectrin based on the kinetics of digestion in the cortical cells (see FIG. 2).

FIG. 10 is a schematic illustration of βII-spectrin degradation pattern by calpain-2 and caspase-3 activated cascades dependent upon the type of neural injury.

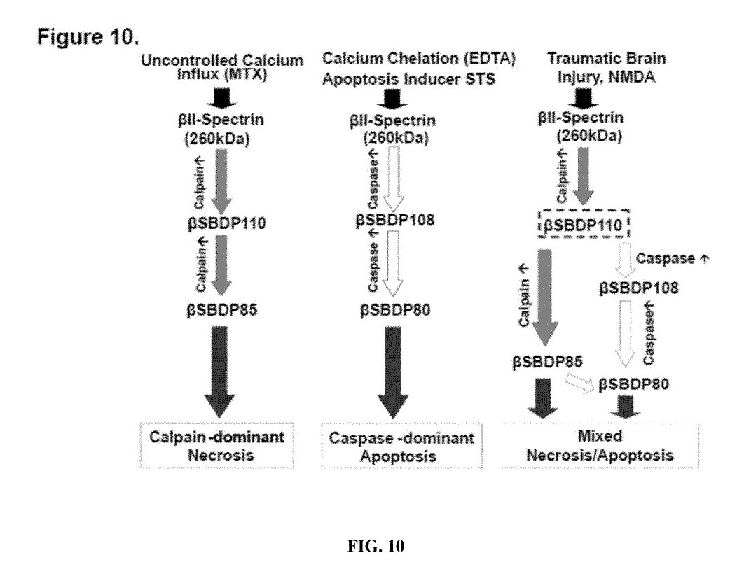

FIG. 11 is an immunostain showing βII-spectrin distribution in rat brain regions 24 hours post-CCI. (A) showing contralateral cortex with intact cytoplasmic βII-spectrin staining; (B) showing the ipsilateral cortex injured region with diffused βII-spectrin staining; (C) showing the contralateral hippocampal (DG region) with intact βII-spectrin; (D) showing the ipsilateral hippocampal (DG region) with decreases βII-spectrin staining.

FIG. 12 is an immunostain showing βII-spectrin distribution in rat pyramidal neurons of cerebral cortex, with βII-spectrin centered in the cytoplasmic/somae area along the plasma membrane of neuronal cells contrastained with neurofilament-L.

FIG. 13 is a schematic view of the in vitro diagnostic device.

### DETAILED DESCRIPTION

The present invention identifies biomarkers that are diagnostic of neural injury, neuronal disorder or neurotoxicity. Detection and quantification of these neurochemical markers helps to determine the severity of the damage, anatomical and cellular pathology of the damage, and appropriate method and course of treatment.

Prior to setting forth the invention, it may be helpful to an understanding thereof to set forth definitions of certain terms that will be used hereinafter.

“Marker” in the context of the present invention refers to a polypeptide (of a particular apparent molecular weight) which is differentially present in a sample taken from patients having neural injury and/or neuronal disorders as compared to a comparable sample taken from control subjects (e.g., a person with a negative diagnosis, normal or healthy subject).

The phrase “differentially present” refers to differences in the quantity and/or the frequency of a marker present in a sample taken from patients having for example, neural injury as compared to a control subject. For example, a marker can be a polypeptide which is present at an elevated level or at a decreased level in samples of patients with neural injury compared to samples of control subjects. Alternatively, a marker can be a polypeptide which is detected at a higher frequency or at a lower frequency in samples of patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in samples of patients' suffering from neural injury, neuronal disorders or neurotoxicity, is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of neural injury and/or neuronal disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without neural injury and/or neuronal disorder. A control amount can be either in absolute amount (e.g., μg/ml) or a relative amount (e.g., relative intensity of signals).

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F (ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region. For the avoidance of doubt, the term “antibody” refers to an antibody that is raised against a particular sequence, immunogen, protein or fragment.

“Immunoassay” is an assay that uses an antibody to specifically bind an antigen (e.g., a marker). The immunoassay is characterized by the use of specific binding properties of a particular antibody to isolate, target, and/or quantify the antigen.

As used herein, the term “in vitro diagnostic” means any form of diagnostic test product or test service, including but not limited to a FDA approved, or cleared, In Vitro Diagnostic (IVD), Laboratory Developed Test (LDT), or Direct-to-Consumer (DTC), that may be used to assay a sample and detect or indicate the presence of, the predisposition to, or the risk of, diseases, disorders, conditions, infections and/or therapeutic responses. In one embodiment, an in vitro diagnostic may be used in a laboratory or other health professional setting. In another embodiment, an in vitro diagnostic may be used by a consumer at home. In vitro diagnostic test comprise those reagents, instruments, and systems intended for use in the in vitro diagnosis of disease or other conditions, including a determination of the state of health, in order to cure, mitigate, treat, or prevent disease or its sequelae. In one embodiment in vitro diagnostic products may be intended for use in the collection, preparation, and examination of specimens taken from the human body. In certain embodiments, in vitro diagnostic tests and products may comprise one or more laboratory tests such as one or more in vitro diagnostic tests. As used herein, the term “laboratory test” means one or more medical or laboratory procedures that involve testing samples of blood, serum, plasma, CSF, sweat, saliva or urine, or other human tissues or substances.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised against marker NF-200 from specific species such as rat, mouse, or human can be selected to obtain only those polyclonal antibodies that are specifically immunoreactive with marker NF-200 and not with other proteins, except for polymorphic variants and alleles of marker NF-200. This selection may be achieved by subtracting out antibodies that cross-react with marker NF-200 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

As used herein, the term “injury or neural injury” is intended to include a damage which directly or indirectly affects the normal functioning of the CNS. For example, the injury can be damage to retinal ganglion cells; a traumatic brain injury; a stroke related injury; a cerebral aneurism related injury; a spinal cord injury, including monoplegia, diplegia, paraplegia, hemiplegia and quadriplegia; a neuroproliferative disorder or neuropathic pain syndrome. Examples of CNS injuries or disease include TBI, stroke, concussion (including post-concussion syndrome), cerebral ischemia, neurodegenerative diseases of the brain such as Parkinson's disease, Dementia Pugilistica, Huntington's disease and Alzheimer's disease, Creutzfeldt-Jakob disease, brain injuries secondary to seizures which are induced by radiation, exposure to ionizing or iron plasma, nerve agents, cyanide, toxic concentrations of oxygen, neurotoxicity due to CNS malaria or treatment with anti-malaria agents, trypanosomes, malarial pathogens, and other CNS traumas.

The terms “patient” or “individual” are used interchangeably herein, and is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

Unless otherwise defined, all technical terms used herein have the same meaning as commonly understood by one of ordinary skill in the art to which this invention pertains. Suitable methods and materials for practicing the invention are described below. The particular embodiments discussed below are intended to be illustrative only and are not intended to be limiting in scope.

### General Biological Methods

Methods involving conventional biological techniques are described herein. For example collection of urine may be done using conventional collection cups or tubes, while sample of saliva may use swabs or tubes, or other such collection devices employed for saliva collection. Such techniques are generally known in the art and are described in detail in methodology treatises such as Molecular Cloning: A Laboratory Manual, 2^{nd} ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g. preparation of antigen-specific antibodies, immunoprecipitation, and immunoblotting) are described, e.g., in Current Protocols in Immunology, ed. Coligan eta al., John Wiley & Sons, New York, 1991; and Methods of Immunological Analysis, ed. Masseyeff et al., John Wiley & Sons, New York, 1992.

### Detecting Neural Injury, Neuronal Disorders or Neurotoxicity

The invention encompasses methods for detecting the presence of the marker βII-spectrin or one of its βII-spectrin breakdown products (βII-SBDPs) in a biological sample as well as methods for measuring the level of such marker in a biological sample. An exemplary method for detecting the presence or absence of βII-spectrin or one of its βII-SBDPs in a biological sample involves obtaining a biological sample from a subject (e.g. human patient), contacting the biological sample with a compound or an agent capable of detecting the marker being analyzed (e.g., an antibody or aptamer), and analyzing binding of the compound or agent to the sample after washing. Those samples having specifically bound compound or agent express of the marker being analyzed.

The biological sample is preferably a biological fluid in communication with the nervous system at the time of injury. These biological fluids include, but are not limited to, cerebrospinal fluid (CSF), blood, plasma, serum, saliva, and urine, as the samples are readily and easily obtained.

A biological sample can be obtained from a subject by conventional techniques. For example, CSF can be obtained by lumbar puncture. Blood can be obtained by venipuncture, while plasma and serum can be obtained by fractionating whole blood according to known methods. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J. Clin. Microbiol. 38: 3892-3895 (2000).

Any animal that expresses the neural proteins, such as for example, those listed in Table 1, can be used as a subject from which a biological sample is obtained. Preferably, the subject is a mammal, such as for example, a human, dog, cat, horse, cow, pig, sheep, goat, primate, rat, mouse and other vertebrates such as fish, birds and reptiles. More preferably, the subject is a human. Particularly preferred are subjects suspected of having or at risk for developing traumatic or non-traumatic nervous system injuries, such as victims of brain injury caused by traumatic insults (e.g. gunshots wounds, automobile accidents, sports accidents, shaken baby syndrome), ischemic events (e.g. stroke, cerebral hemorrhage, cardiac arrest), spinal cord injury, neurodegenerative disorders (such as Alzheimer's, Huntington's, and Parkinson's diseases; Prion-related disease; other forms of dementia, and spinal cord degeneration), epilepsy, substance abuse (e.g., from amphetamines, methamphetamine/Speed, Ecstasy/MDMA, or ethanol and cocaine), and peripheral nervous system pathologies such as diabetic neuropathy, chemotherapy-induced neuropathy and neuropathic pain, peripheral nerve damage or atrophy (ALS), multiple sclerosis (MS).

### Markers of Calpain-2 and Caspase-3 Activation

The method of the invention features a step of detecting in a biological sample the presence or amount of at least one marker selected from βII-spectrin and a βII-SBDP generated from proteolytic cleavage of βII-spectrin by at least one protease selected from the group consisting of calpain-2 and caspase-3. βII-SBDPs generated from the proteolytic cleavage of 3II-spectrin by calpain-2 include βII-SBDP-85 (85 kDa) and βII-SBDP-110 (110 kDa). βII-SBDPs generated from the proteolytic cleavage of βII-spectrin by caspase-3 include βII-SBDP-80 (80 kDa) and βII-SBDP-108 (108 kDa). It should be appreciated that the un-fragmented, or intact, βII-spectrin has a molecular weight of 260 kDa. Accordingly, where a fragment of interest has a certain molecular weight, there remains one or more additional protein fragments that may also be detected through similar means and through using antibodies which either interact with those fragments globally or specifically and independently interact with those specific fragments.

### Detection of Biomarkers

The biomarkers of the invention can be detected in a sample by any means. Methods for detecting the biomarkers are described in detail in the materials and methods and Examples which follow. For example, immunoassays include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, fluorescent immunoassays and the like. Such assays are routine and well known in the art (see, e.g., Ausubel et al, eds, 1994, Current Protocols in Molecular Biology, Vol. 1, John Wiley & Sons, Inc., and New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding an antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen (i.e. neural biomarker), coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Ten, eds., 7th ed. 1991); and Harlow & Lane, supra. The detection and quantitation of biomarkers is described in detail in the Examples which follow.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include cerebrospinal fluid, blood, serum, plasma, neuronal cells, tissues, urine, tears, saliva etc. In a preferred embodiment, the biological fluid comprises cerebrospinal fluid. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Correlating Marker Expression with Neural Injury, Neuronal Disorder, or Neurotoxicity

The invention provides a step of correlating the presence or amount of βII-spectrin and/or one or more of βII-SBDPs in a biological sample with the severity and/or type of nerve cell (or other βII-spectrin expressing cell) injury. The amount of βII-spectrin and/or its βII-SBDPs in the sample directly relates to the severity of nerve tissue injury as a more severe injury damages a greater number of nerve cells which in turn causes a larger amount of βII-spectrin and/or its βII-SBDPs to accumulate in the sample. Examining which βII-SBDPs are present in the sample and their amounts will help determine whether cellular death is primarily apoptic or necrotic. Apoptic cell death preferentially activates caspase, while necrotic cell death preferentially activates calpain. Because calpain-2 and caspase-3 βII-SBDPs may be distinguished, measurement of these markers indicates the type of cell damage in a subject. For example, necrosis-induced calpain-3 activation results in production of βII-sSBDP-85 and βII-SBDP-110, while apoptosis-induced caspase-2 results in production of βII-SBDP-80 and βII-SBDP-108. The results of such a test can help a physician determine whether the administration of calpain and/or caspase inhibitors in order to limit βII-spectrin degradation might benefit an injured patient.

### Kits

The invention also provides a kit for analyzing cell damage in a subject. The kit includes: (a) a substrate for holding a biological sample isolated from a human subject suspected of having a damage nerve cell, the biological sample being a fluid in communication with the nervous system of the subject prior to being isolated from the subject; (b) an agent that specifically binds at least one marker selected from βII-spectrin and a βII-SBDP generated from proteolytic cleavage of βII-spectrin by at least one protease selected from the group consisting of calpain-2 and caspase-3; and (c) printed instructions for reacting the agent with the biological sample or a portion of the biological sample to detect the presence or amount of the at least one marker in the biological sample.

In the kit, the biological sample can be CSF, blood, plasma, serum, saliva, or urine, and the agent can be an antibody, aptamer, or other molecule that specifically binds at least one of βII-spectrin, βII-SBDP-80, β1I-SBDP-85, βII-SBDP-108, and βII-SBDP-110. Suitable agents are described above. The kit can also include a detectable label such as one conjugated to the agent, or one conjugated to a substance that specifically binds to the agent (e.g., a secondary antibody).

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### In Vitro Diagnostic Devices

In another embodiment, the invention provides an in vitro diagnostic device to measure biomarkers that are indicative of neuro-regeneration. Preferably, the biomarkers are proteins, fragments or derivatives thereof, and are associated with neuro-regeneration and improved cognitive function.

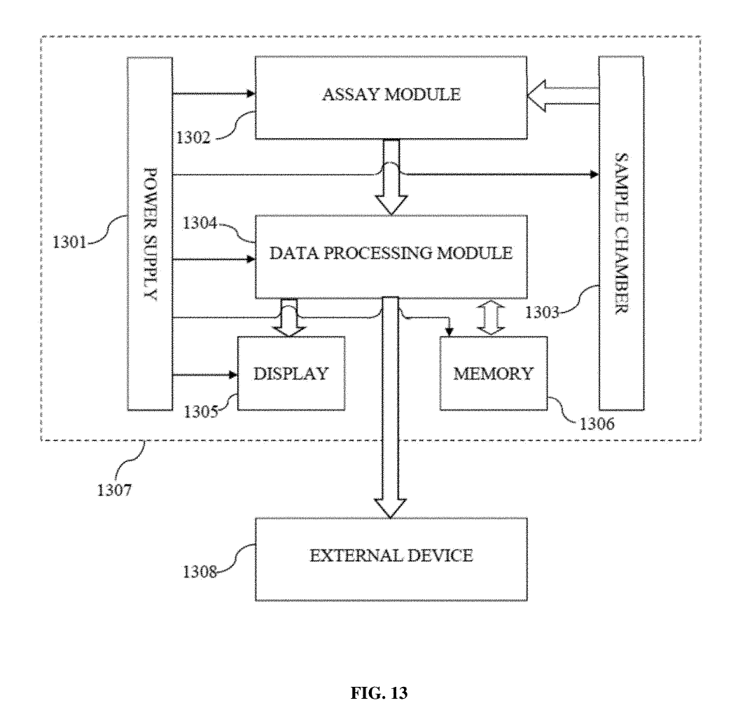

FIG. 13 schematically illustrates the inventive in vitro diagnostic device. An inventive in vitro diagnostic device comprised of at least a sample collection chamber 1303 and an assay module 1302 used to detect biomarkers of neural injury or neuronal disorder. The in vitro diagnostic device may comprise of a handheld device, a bench top device, or a point of care device.

The sample chamber 1303 can be of any sample collection apparatus known in the art for holding a biological fluid. In one embodiment, the sample collection chamber can accommodate any one of the biological fluids herein contemplated, such as whole blood, plasma, serum, urine, sweat or saliva.

The assay module 1302 is preferably comprised of an assay which may be used for detecting a protein antigen in a biological sample, for instance, through the use of antibodies in an immunoassay. The assay module 1302 may be comprised of any assay currently known in the art; however the assay should be optimized for the detection of neural biomarkers used for detecting neural injury or neuronal disorder in a subject. The assay module 1302 is in fluid communication with the sample collection chamber 1303. In one embodiment, the assay module 1302 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. In one embodiment a colorimetric assay may be used which may comprise only of a sample collection chamber 1303 and an assay module 1302 of the assay. Although not specifically shown these components are preferably housed in one assembly 1307. In one embodiment the assay module 1302 contains an agent specific for detecting βII-spectrin or one of its βII-spectrin breakdown products (βII-SBDPs). The assay module 1302 may contain additional agents to detect additional biomarkers, as is described herein.

In another preferred embodiment, the inventive in vitro diagnostic device contains a power supply 1301, an assay module 1302, a sample chamber 1303, and a data processing module 1305. The power supply 1301 is electrically connected to the assay module and the data processing module. The assay module 1302 and the data processing module 1305 are in electrical communication with each other. As described above, the assay module 1302 may be comprised of any assay currently known in the art; however the assay should be optimized for the detection of neural biomarkers used for detecting neural injury or neuronal disorder in a subject. The assay module 1302 is in fluid communication with the sample collection chamber 1303. The assay module 1302 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 1303 and assayed by the assay module 1302 detecting for a biomarker of neural injury or neuronal disorder. The measured amount of the biomarker by the assay module 1302 is then electrically communicated to the data processing module 1304. The data processing 1304 module may comprise of any known data processing element known in the art, and may comprise of a chip, a central processing unit (CPU), or a software package which processes the information supplied from the assay module 1302.

In one embodiment, the data processing module 1304 is in electrical communication with a display 1305, a memory device 1306, or an external device 1308 or software package (such as laboratory and information management software (LIMS)). In one embodiment, the data processing module 1304 is used to process the data into a user defined usable format. This format comprises of the measured amount of neural biomarkers detected in the sample, indication that a neural injury or neuronal disorder is present, or indication of the severity of the neural injury or neuronal disorder. The information from the data processing module 404 may be illustrated on the display 1305, saved in machine readable format to a memory device, or electrically communicated to an external device 1308 for additional processing or display. Although not specifically shown these components are preferably housed in one assembly 1307.

In one embodiment, the methods and in vitro diagnostic tests and products described herein may be used for the detection of neuro-regeneration or improved cognitive function of a patient. In yet another embodiment, the methods and in vitro diagnostic tests described herein may indicate diagnostic information to be included in the current diagnostic evaluation in patients suspected of having neural injury or neuronal disorder.

In one embodiment, an in vitro diagnostic test may comprise one or more devices, tools, and equipment configured to hold or collect a biological sample from an individual. In one embodiment of an in vitro diagnostic test, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. Such reagents and solutions for nucleotide collecting, stabilizing, storing, and processing are well known by those of skill in the art and may be indicated by specific methods used by an in vitro diagnostic test as described herein. In another embodiment, an in vitro diagnostic test as disclosed herein, may comprise a micro array apparatus and reagents, a flow cell apparatus and reagents, a multiplex nucleotide sequencer and reagents, and additional hardware and software necessary to assay a genetic sample for certain genetic markers and to detect and visualize certain biological markers.

Incorporation of these biomarkers in an in vitro diagnostic device enables for a hand held, bench top or point of care (POC) diagnostic device which enables the accurate and rapid diagnosis of a neural regeneration.

### EXAMPLES

### Materials and Methods

### Abbreviations:

AEB SF, 4-(2-aminoethyl)-benzenesulfonylflouride; EDTA, ethylenediaminetetraacetic acid; EGTA, ethylenebis(oxyethylenenitrilo) tetra acetic acid; DMEM, Dulbecco's modified Eagle's medium; BSA, bovine serum albumin; DPBS, Dulbecco's phosphate buffered saline; DTT, dithiothreitol; FDA, fluorescein diacetate; MTX, maitotoxin; NMDA, N-methyl-D-aspartate; STS, staurosporine; HBSS, Hanks' balanced salt solution; MAP-2, microtubule associated protein-2; PI, propidium iodide; PMSF, phenylmethylsulfonyl fluoride; SDS, sodium dodecyl sulfate; TEMED, N,N,N′,N′-tetramethyletheylenediamine; Calpinh-II, calpain inhibitor II (N-acetyl-Leu-Leu-methioninal); Z-D-DCB, pan-caspase inhibitor(carbobenzoxy-Asp-CH₂—OC (O)-2-6-dichlorobenzene); PBS, phosphate buffered saline; TLCK, Na-p-tosyl-L-Lysine chloro methyl; TPCK, N-tosyl-L-phenylalanine chloromethyl ketone.

### In Vivo Model of Experimental Traumatic Brain Injury (TBI)

A controlled cortical impact (CCI) device is used to model TBI in rats as described previously (Pike et al., 1998). Adult male (280-300 g) Sprague-Dawley rats (Harlan, Indianapolis, U.S.A.) are anaesthetized with 4% isofluorane in a carrier gas of O2/N2O, 1:1 (4 min duration) followed by maintenance anesthesia with 2.5% isofluorane in the same carrier gas. Core body-temperature is monitored continuously by a rectal thermistor probe and maintained at 37±1° C. by placing an adjustable temperature controlled heating pad beneath the rats. Animals are supported in a stereotactic frame in a prone position and secured by ear and incisor bars. A midline cranial incision is made, the soft tissues revealed, and a unilateral (ipsilateral to the site of impact) craniotomy (7 mm diameter) is performed adjacent to the central suture, midway between bregma and lambda. The dura mater is kept intact over the cortex. Brain trauma is produced by impacting the right cortex (ipsilateral cortex) with a 5 mm diameter aluminum impactor tip (housed in a pneumatic cylinder) at a velocity of 3.5 m/s with a 1.6 mm (severe) compression and 150 ms dwell-time (compression duration). These injuries are associated with local cortical contusion and diffuse axonal damage. Velocity is controlled by adjusting the pressure (compressed N2) supplied to the pneumatic cylinder. Velocity and dwell-time are measured by a linear velocity displacement transducer (Lucas Shaevitz® model 500 HR, Detroit, Mich., U.S.A.) that produced an analogue signal which is recorded by a storage-trace oscilloscope (BK Precision, model 2522B, Placentia, Calif., U.S.A.). Sham-injured control animals undergo identical surgical procedures but do not receive an impact injury. Pre- and post-injury management are in compliance with guidelines set forth by a local Institutional Animal Care and Use Committee (IACUC) and the NIH (National Institutes of Health) guidelines detailed in the Guide for the Care and Use of Laboratory Animals.

### Cortical and Hippocampal Tissue Collection and Protein Extraction

At the appropriate time-points (2, 6, 24 hrs, and 3, 5, 7, 14 days) post CCI, the animals are anaesthetized and immediately sacrificed by decapitation. Brains are immediately removed, rinsed with ice-cold PBS and halved. Four different brain regions in right hemispheres (cerebrocortex, subcortical white matter, hippocampus and corpus callosum) are rapidly dissected, rinsed in ice cold PBS, snap-frozen in liquid nitrogen and frozen at −80 ° C. until use. For the left hemisphere, the same tissue as the right side is collected. For Western blot analysis, targeted brain samples are pulverized to a fine powder with a small mortar/pestle set over solid CO2. The pulverized tissue powder is then lysed for 90 min at 4.0 with 50 mM Tris (pH 7.4), 2 mM EDTA, 1% (v/v) Triton X-100 and 1 mM DTT (dithiothreitol) and lx tablet protease inhibitor cocktail (Roche Biochemicals, Indianapolis, IN). The brain lysate is then centrifuged at 15000×g for 15 min at 4° C., to clear and remove insoluble debris, snap-frozen and stored at −85° C. until further use.

Primary Cerebrocortical Culture

All cultures are prepared in quadruplicate. Cerebrocortical cells harvested from 1-day old Sprague-Dawley rat brains are plated on poly-L-lysine coated 6-well culture plates (Erie Scientific, Portsmouth, N.H., USA) according to a previously cited method (Nath et al., 2000) at a density of 4.36×105 cells/mL. Cultures are maintained in Dulbecco's modified Eagle's medium (DMEM) with 10% fetal bovine serum in a humidified incubator in an atmosphere of 10% CO2 at 37° C. After 5 days in culture, the media is changed to DMEM with 5% horse serum. Subsequent media changes are performed three times a week. Experiments are performed on days 10 to 11 in vitro when astroglia have formed a confluent monolayer beneath morphologically mature neurons. All animal studies conform to the guidelines outlined in the Guide for the Care and Use of Laboratory Animals from the National Institutes of Health and were approved by the local IACUC.

### Neurotoxin Challenges and Pharmacologic Intervention

In addition to untreated controls, the following conditions are used: NMDA (N-methyl-D-aspartate; 300 μM; Sigma-Aldrich, St. Louis, Mo.O) for 3-24 hrs as an excitotoxic effect (Nath et al., 2000); apoptotic inducers STS (staurosporine) (0.5 μM; Sigma, St. Louis, Mo., U.S.A.) that activates calpain and caspase-3 for 24 hrs (Zhang et al., 2009); the Ca2+chelator EDTA (2 mM; Sigma-Aldrich, St. Louis, Mo.) for up to 24 hr as a caspase-dominant challenge (Chiesa et al., 1998; Waterhouse et al., 1996). For pharmacological intervention, cultures are pretreated 1 hr before the STS, EDTA or NMDA challenge with 30 μM of the calpain inhibitor SNJ-1945 (Senju Pharmaceuticals, Kobe, Japan) (Koumura et al., 2008; Shirasaki et al., 2005), or with 20 μM the caspase-3 inhibitor IDN-6556 (Baskin-Bey et al., 2007; Hoglen et al., 2007; Pockros et al., 2007; Poordad, 2004). Cells are collected and lysed with the same lysis buffer as described above.

### Cell Lysate Collection and Preparation

Primary neuronal cell cultures are harvested and lysed for 90 min at 4° C. with 50 mM Tris (pH 7.4), 2 mM EDTA, 1% (v/v) Triton X-100, 1 mM DTT, 1× protease inhibitor cocktail (Roche Biochemicals, Indianapolis, Ind.). The neuronal lysates are then centrifuged at 15000 g for 5 min at 4° C. to clear and remove insoluble debris, snap-frozen, and stored at −85° C. until use.

### SDS-Polyacrylamide Gel Electrophoresis and Electrotransfer

Protein concentrations of culture lysates are determined by bicinchoninic acid microprotein assays (Pierce Inc, Rockford, Ill., USA) with albumin standards. Protein balanced samples are prepared for sodium dodecyl sulfate-polyacrylamide gel electrophoresis (SDS-PAGE) in eight-fold loading buffer containing 0.25 M Tris (pH 6.8), 0.2 M DTT, 8% SDS, 0.02% bromophenol blue, and 20% glycerol in distilled H2O. Twenty micrograms (20 μg) of protein per lane are routinely resolved by SDS-PAGE on 6% Tris/glycine gels (for spectrin protein) and/or 10-20% Tris/glycine gels for 2 hr at 120 V. Following electrophoresis, separated proteins are laterally transferred to polyvinylidene fluoride (PVDF) membranes in a transfer buffer containing 0.50 M glycine, 0.025 M Tris-HC1 (pH 8.3) and 10% methanol at a constant voltage of 20 V for 2 hr at 4° C. in a semi-dry transfer unit (Bio-Rad, Hercules, Calif.).

### Calpain-2 and Caspase-3 Digestion of Naïve Brain Lysate and Purified Proteins

For these experiments, brain tissue (cortex and hippocampus) collection and preparation are the same as described above. Owing to the need for in vitro protease-mediated digestion, protease inhibitor cocktail is not used. In vitro protease digestion of naïve rat hippocampus lysate (30 mg) or purified recombinant human βII-spectrin (Panvera Co., Madison, Wis., U.S.A.) with purified proteases, human calpain-2 (BD Bioscience; NJ, Catalogue no. 208715, 1 mg/ml), and caspase-3 (BD Bioscience, NJ, 1 unit/ml) is performed in a buffer containing 100 mM Tris/HCl (pH 7.4) and 20 mM DTT. For calpain-2, 2 mM CaCl2 is also added, and then incubated at room temperature (25° C.) for 30 min. In addition, 2 mM EDTA is added for caspase-3 and the mixture was incubated at 37° C. for 4 hrs. The protease reaction is stopped by the addition of PAGE-sample buffer.

### Immunoblotting Technique

Tissue samples (20 μg) are subjected to electrophoresis, equal volumes of samples for SDS/PAGE are prepared in a 2×-fold loading buffer [0.25 M Tris (pH 6.8), 0.2 M DTT, 8% SDS, 0.02% bromophenol blue and 20% glycerol in distilled H2O]. Gels are run at 120 V for 2 hr in a mini-gel unit (Invitrogen Life). Protein bands are transferred to PVDF membrane on a semi-dry Trans-blot unit (Bio-Rad, Hercules, Calif.) at 20 V for 2 hrs. After electrotransfer, blotting membranes are blocked for 1 hr at ambient temperature in 5% non-fat milk in TBST [20 mM Tris/HCl (pH 7.4), 150 mM NaCl and 0.05% (w/v) Tween-20], then incubated with the primary monoclonal antibody in TBST/5% milk. Primary antibodies to be used include mouse anti all-spectrin (Affinity Res. Prod. Nottingham, UK), mouse monoclonal anti βII-spectrin (BD Transduction Laboratories, USA; cat # 612563) and rabbit anti βIII-spectrin (BETHYL Laboratories, Inc. TX, cat # A310-367A). The blots are then washed 3 times for 15 min with TBST and exposed to biotinylated secondary antibodies (Amersham Biosciences, U.K.) followed by a 30 min incubation with streptavidin-conjugated alkaline phosphatase. Colorimetric development is performed with a one-step 5-bromo-4-chloro-3-indolyl phosphate-reagent (Sigma-Aldrich, St. Louis, Mo.). The molecular weights of intact proteins and their potential BDPs are assessed by running along-side rainbow colored molecular weight standards (Amersham Biosciences, U.K.)

### Immunohistochemical Experiments

Immunohistochemical (IHC) analysis is performed on paraffin-embedded 6 μm rat brain sections. Slides are deparaffinized, incubated for 10 min at 95° C. in Trilogy solution (Cell Marque, Hot Springs, AK) for antigen retrieval and blocked for endogenous peroxides with 3% hydrogen peroxide. For Immunoflorescent double-labeling experiment the sections are additionally incubated with 2% normal goat serum. Then these sections are incubated with Anti-3-Spectrin II (BD Transduction Laboratories) and Anti-Neurofilaments L (Cell signaling) overnight at 4C° followed by treatments with secondary anti-mice Alexa Fluor 555 Conjugate (Invitrogen) and Anti-rabbit Alexa Fluor 488 (Invitrogen) diluted in 2% goat serum for 2 h at RT. Then the sections are washed with PBS, mounted, air dried and coverslipped with mounting medium with DAPI (Vector). Staining is examined using fluorescence microscope (Leica). For colorimetric DAB staining the sections are incubated with Anti-β-Spectrin II (BD Transduction Laboratories) overnight at 4 C° followed by treatments with secondary goat anti-mice HRP (Dako). The staining is visualized with 3,3′-diaminobenzidine (DAB) (Dako, Carpinteria, Calif.) for brown color development. Then the sections are counterstained with Hematoxylin (Dako, Carpinteria, Calif.). In control experiments primary antibodies are omitted. Sections are finally washed with PBS, mounted, air dried and coverslipped with mounting medium Aquamount (Dako). Staining is examined using Aperio ScanScope GL.

### Statistical Analyses

Semi-quantitative evaluation of protein and BDP levels is performed via computer-assisted densitometric scanning (Epson XL3500 high resolution flatbed scanner) and image analysis using NIH ImageJ densitometry software (version 1.6, NIH, Bethesda, Md.). Changes in any outcome parameter are compared with the appropriate control group. Thus, magnitude of change from control in one model system is directly compared with magnitude of change from any other model system. Six replicate results are evaluated by t-test and ANOVA and post-hoc Tukey tests. A value of p<0.05 is considered significant.

### Example 1

### Tissue Distribution of βII-spectrin Protein Expression.

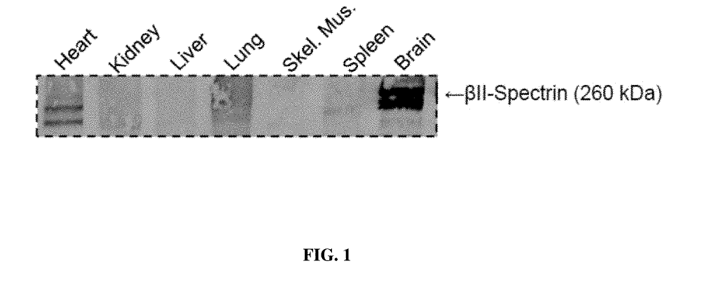

βII-spectrin protein expression is examined for brain specificity in a rat tissue panel by Western blotting technique. βII-spectrin is found to be highly expressed in the brain with only minute amounts being found in other organs (e.g. lung, kidney and heart). (FIG. 1).

### Example 2

### Detection of Calpain-2 and Caspase-3 βIIsBDPs Following In Vitro Neuronal Insult

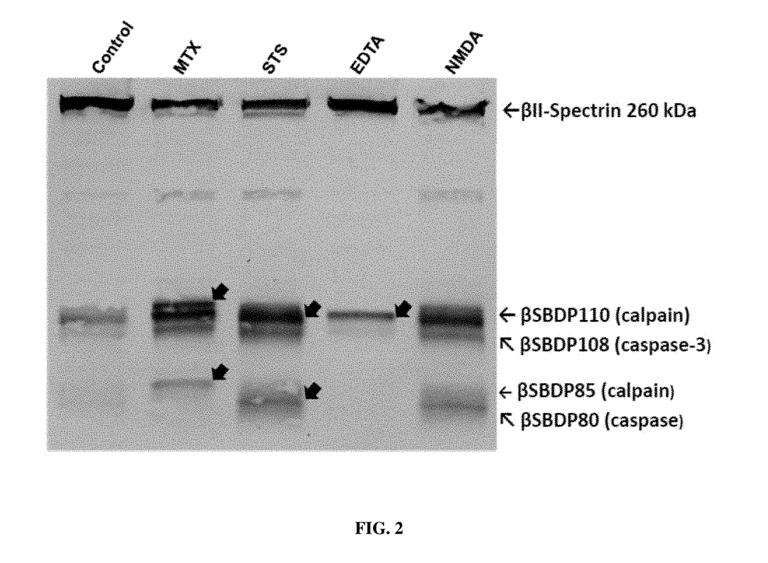

Rat cerebrocortical cultures are prepared as described above and left untreated (serving as control) or treated with either maitotoxin (MTX) for 3 hours or Ca²⁺ chelator (EDTA), to assess necrotic or apoptic cell death, respectively. Alternatively, neuronal cultures are treated with staurosporine (STS) for 24 hours or with NMDA. (FIG. 2)

Control neuronal cells show healthy cell body and well-defined neurite network under microscope. In contrast, significant degeneration is observed in soma and neuritis in the treated neuronal cultures (MTX at 3 hours, STS at 24 hours, EDTA at 24 hours, NMDA at 24 hours). Specifically, with the NMDA treatment, the 260 kDa βII-spectrin is significantly degraded into multiple fragments including a dominant signal of calpain-mediated βII-spectrin breakdown product (βII-SBDP) of 110 kDa and 85 kDa, with minimal caspase mediated βII-SBDP of 108 kDa and 80 kDa. This is followed by treating the cultures with another apoptosis inducer, STS, where two prominent βII-SBDP bands of 108 kDa and 80 kDa are observed. When the neuronal culture is treated with another apoptosis inducing EDTA, βII-spectrin truncation pattern reveals weaker βII-SBDP of 108 kDa and a minimal βII-SBDP of 80 kDa. Under necrotic challenge with MTX, there is strong βII-SBDPs of 110 kDa and 85 kDa bands.

The results of this study demonstrate differential βII-spectrin proteolytic vulnerability after apoptotic, necrotic or excitotoxic challenges resulting in calpain and/or caspase specific βII-SBDPs as shown in FIG. 2.

### Example 3

Effects of Calpain-2 and Caspase-3 inhibitors on βII-SBDP Pattern

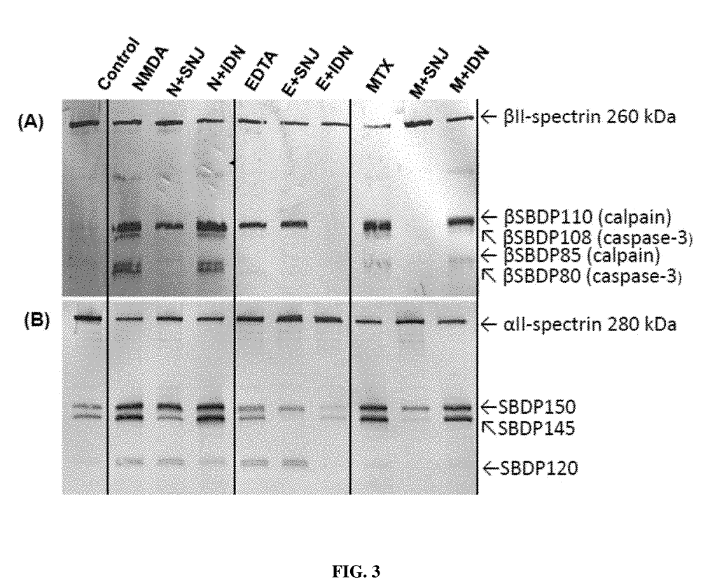

Rat cerebrocortical cell cultures are prepared as described above and treated with calpain inhibitor SNJ-1945 and caspase-3 inhibitor IDN-6556 along with different neurotoxic paradigms (FIG. 3). As shown in FIG. 3A, the cerebrocortical neuronal cultures are either untreated (control) or subjected to EDTA alone, EDTA with caspase-3 inhibitor, or EDTA with calpain-2 inhibitor. Western blot analysis shows that there are strong βII-SBDP 108 kDa/weak 80 kDa bands in EDTA alone and (EDTA+SNJ-1945) lanes, but there are no βII-SBDPs in control and EDTA with IDN-6556 lanes. This was compared to aII-spectrin breakdown pattern which confirmed the presence of caspase-mediated SBDP-120 (120 kDa) in EDTA, and its absence in the control and in the (EDTA+IDN-6556) lanes as shown in FIG. 3B.

Taken together, the data indicates that the βII-SBDPs-108 kDa/80 kDa are caspase-3 specific and comprise the prominent degradation bands seen in an apoptotic event. Similarly, the cerebrocortical cultures are subjected to 0.5 nM MTX treatment alone for 3 hrs or (MTX+20 μM IDN-6556) or (MTX+30 μM SNJ-1945). βII-SBDPs of 110 kDa and 85 kDa are observed in MTX alone and MTX with the caspase inhibitor IDN-6556 lanes. However, there are no βII-SBDPs observed in the control and MTX with SNJ-1945 lanes which suggests that βII-SBDPs of 110 kDa and 85 kDa are both calpain-induced, since MTX would induce necrotic injury (FIG. 3A). This is confirmed by the αII-spectrin breakdown pattern which indicates the presence of the prominent calpain-mediated SBDP-145 kDa band in the MTX treatment and its absence in the control and SNJ-1945 lanes as shown in FIG. 3B. Finally, cerebrocortical neuron cultures are challenged with either 300 μM NMDA alone, (NMDA+20 μM IDN-6556) or (NMDA+30 μM SNJ-1945). NMDA treatment exhibits an excitotoxic effect with mixed necrotic and apoptotic phenotypes on the neuronal cells. Western blot analysis reveals the presence of all the βII-SBDPs (110, 108, 85 and 80 kDa) in the NMDA lane. There are similar βII-SBDPs pattern in NMDA with IDN-6556 to those observed in the NMDA lane, but much weaker. In contrast, there is only βII-SBDP-108 kDa band in NMDA with 30 μM SNJ-1945 lane (FIG. 3A). Consistent to the aforementioned data, when established calpain/caspase dual-substrate αII-spectrin is probed (Wang, 2000), it clearly shows that NMDA-yields prominent calpain-mediated SBDP-150/SBDP-145, with minor bands of caspase-3-mediated SBDP-120 (FIG. 3B, bottom panel). These fragments are strongly inhibited with their respective protease inhibitors (SNJ-1945 and IDN-6556). The data suggests that in excitotoxic conditions there is concomitant activation of calpain and caspase-3 resulting in the production of all βII-SBDPs (110, 108, 85 and 80 kDa) as shown in FIG. 3.

### Example 4

### Detection of Calpain-2 and Caspase-3 βIIsBDPs Following In Vivo Neuronal Insult

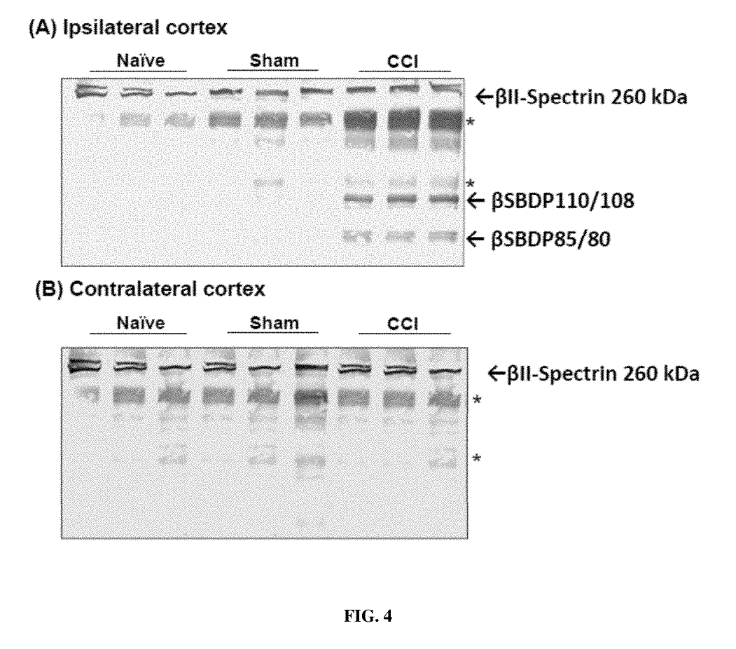

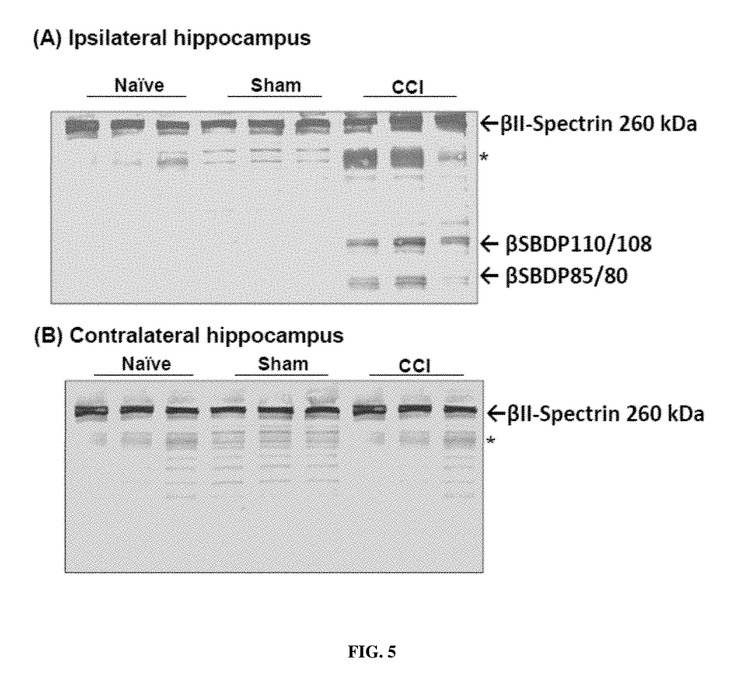

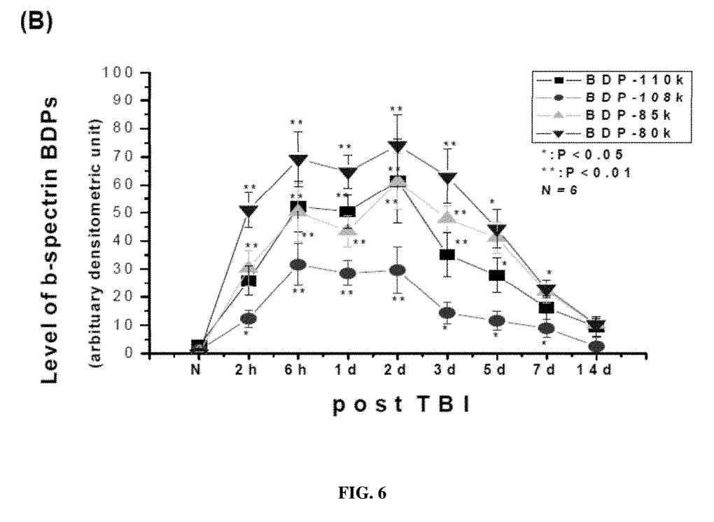

TBI is induced in rodents as described above. Following TBI or sham operation, samples of cortical and hippocampal tissues are harvested and analyzed for presence of calpain-2 specific and caspase-3 specific βII-SBDPs (FIGS. 5 and 6). In the ipsilateral cortex at 48 hours after TBI, βII-spectrin is degraded, generating the caspase/calpain signature βII-SBDPs including the 110, 108, 85, and 80 kDa fragments, thus indicating activation of calpain-2 and caspase-3 in the TBI group (FIG. 4A). No βII-SBDPs bands are found in ipsilateral naïve samples and minimal βII-SBDPs are observed in sham samples as shown by FIG. 4A. In addition to the contralateral cortex, no βII-spectrin proteolysis is observed in all three groups (FIG. 4B). Similar analysis is performed on the hippocampal brain region (ipsilateral vs. contralateral) in the three groups of naïve, sham, and CCI animals 48 hours after surgery (FIGS. 5A and 5B). The βII-SBDP pattern observed in the ipsilateral region of the hippocampus at 48 hours after TBI is similar to those observed in the cortical region of the brain (FIG. 5A). No βII-SBDPs are identified in the contralateral region of the hippocampus in control samples but traces are observed in TBI-injured samples (FIG. 5B). The results of this study demonstrate that βII-spectrin generates sustained and specific βII-SBDPs after acute brain insult.

### Example 5

### Time Course Detection of Calpain-2 and Caspase-3 βII-SBDPs Following In Vivo Neuronal Insult

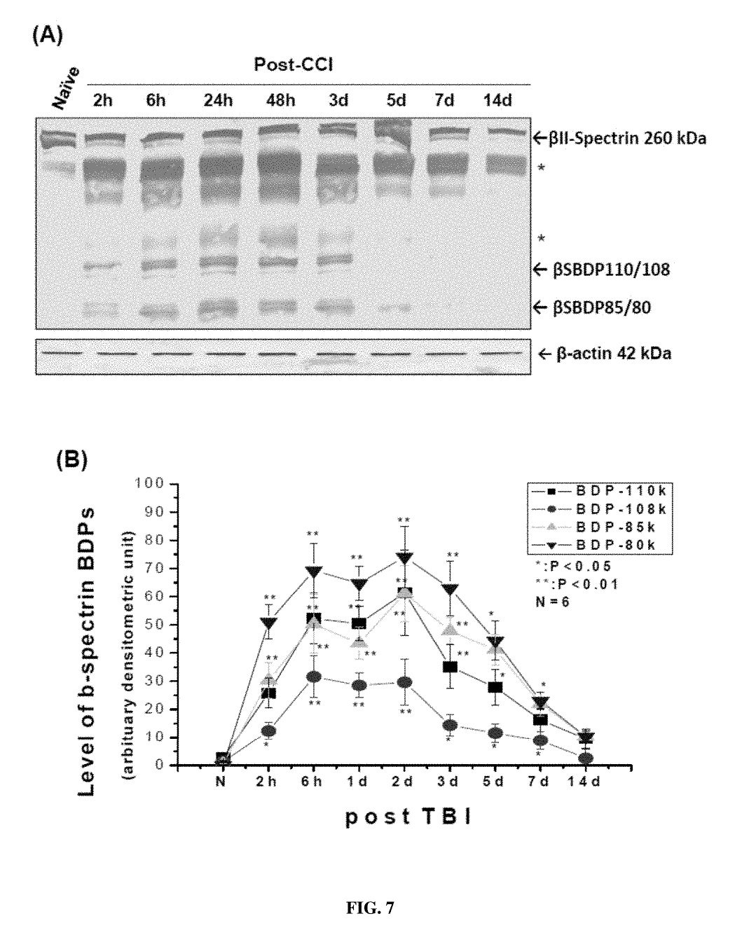

TBI is induced in rodents and samples are harvested as described above. Immunoblots reveal that βII-SBDPs, including fragments of 110, 108, 85, and 80 kDa, accumulate in the ipsilateral cortex at different time points after TBI, peaking at 6 hours after TBI and lasting up to 72 hours, followed by graduate decrease and disappearance after 7 to 14 days (FIGS. 6A and 6B). 110 and 108 kDa βII-SBDPs sustain their presence until day 5, compared to 80/85 kDa βII-SBDPs which last until day 7. The temporal pattern of βII-SBDPs in the ipsilateral hippocampus at different time points post-TBI (FIGS. 7A and 7B) is similar to those in the cortex (FIGS. 7A and 7B). The 110/108 kDa βII-SBDPs sustain their presence until day 3 compared to the 80/85 kDa βII-SBDPs which last until day 5.

### Example 6

Comparison of βII-spectrin Proteolytic Fragmentation After Brain Cortex Digestion with Calpain-2 and Caspase-3 Proteases.

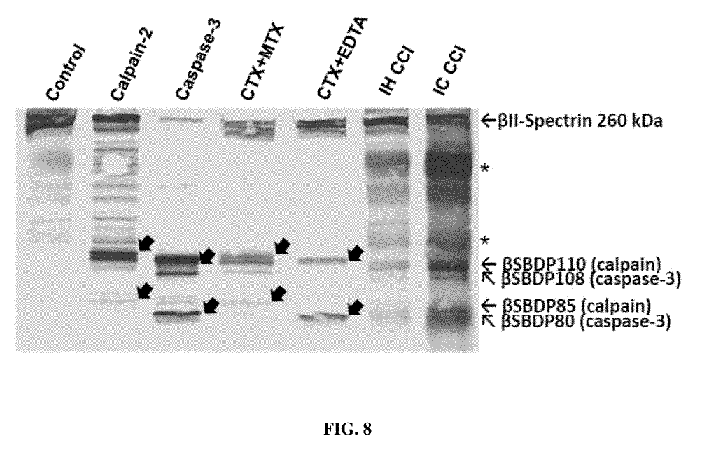

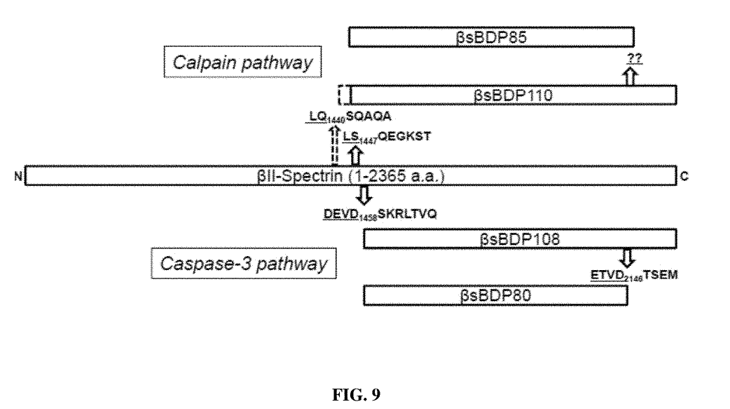

To further validate the fidelity and specificity of βII-SBDPs identified both in vivo and in vitro, cortical tissue lysates are subjected to either calpain-2 or caspase-3 digestion. βII-SBDPs patterns are compared to CCI samples and brain lysates treated with MTX and EDTA as controls for necrotic and apoptotic cell injury, respectively (FIG. 8). Results indicate that the intact 260 kDa βII-spectrin is degraded in vitro into the prominent 108 and 80 kDa βII-SBDPs after caspase-3 digestion. Calpain digestion generates the prominent 110 and 85 kDa βII-SBDPs in addition to a number of non-specific high molecular bands (FIG. 8). EDTA and MTX treatments generated the 108/80 kDa and 110/85 kDA βII-SBDPs, respectively mirroring the results of the caspase/calpain digestion, comparable to the in vitro digested brain samples (FIG. 8). The results suggest βII-spectrin proteolytic fragments are generated via the simultaneous cleavage by caspase-3 and calpain-2 proteases to produce specific fragmentation patterns comparable to those generated in in vitro cell cultures as well as the TBI condition. Putative caspase-3/calpain-2 cleavage sites of βII-spectrin (previously documented by Glantz et al., 2007; Wang et al., 1998) match with the kinetics and pattern of βII-spectrin digestion in the cortical cells and post-TBI in vivo (FIG. 9).

### Example 7

### Detection of βII-Spectrin Localization and Distribution Post-TBI

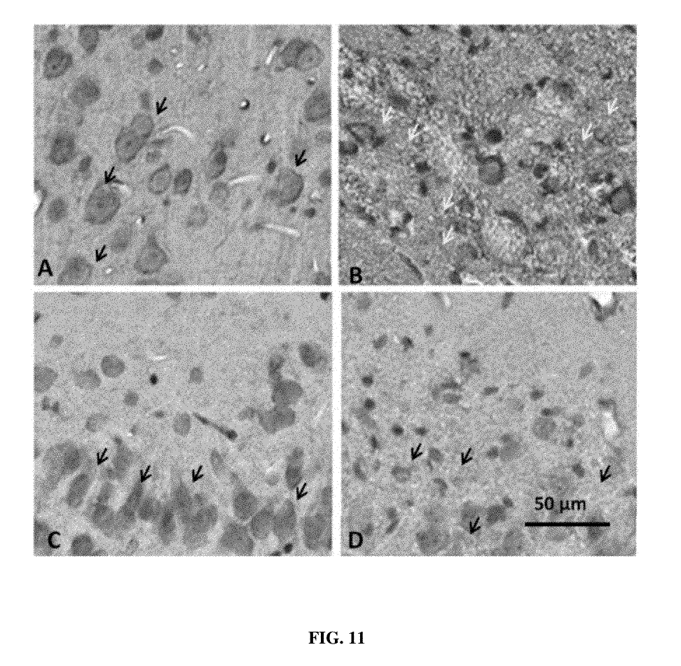

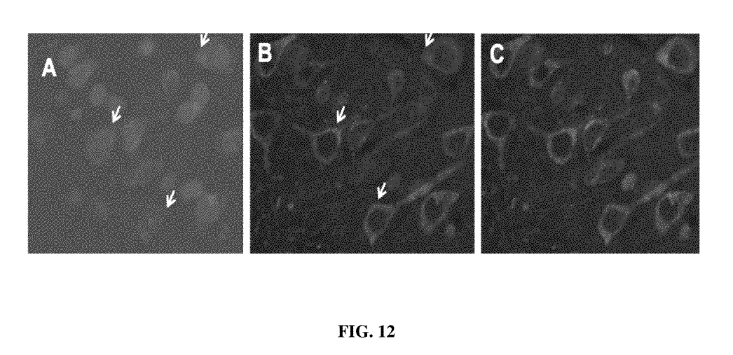

Immunohistochemistry (IHC) expression of βII-spectrin is investigated in the pyramidal neurons of the cerebral cortex (FIG. 12). βII-spectrin is shown to be centered in the cytoplasmic/somae area along the plasma membrane of neuronal cells contrastained with neurofilament-L. These results are in contrast to previously published data showing that βII-spectrin is present in the axons and dendrites compared to other isoforms (Ivy et al., 1988; Ursitti et al., 2001). IHC is also performed on TBI animals to evaluate distribution of βII-spectrin post-injury. IHC data shows an intact neuronal distribution of βII-spectrin in the cortex/hippocampus brain region while ipsilateral injured region reflects a diffused elevated immunostaining pattern of βII-spectrin suggestive of degradation after TBI event (FIG. 11).

### Example 8

Detection of βII-spectrin and βII-SBDPs in CSF of human TBI.

Accumulation of novel markers (βII-spectrin and βII-SBDPs) is analyzed in samples of human CSF taken at 24 hr after nerve cell damage, neural injury, or a neurotoxic event which are stored and transferred using conventional means currently known in the art. The samples are subjected to diagnostic methods known, such as Western Blot or ELISA, and examined for βII-spectrin or any of the βII-SBDPs. Levels of βII-spectrin are found to be much lower in the injured patients than in the control patients while levels of the analyzed βII-SBDPs are found to be increased. This data demonstrates that after TBI, neural proteins accumulate in human CSF in sufficient levels to be easily detectable on Western blots or by other immunoassays such as ELISA.

### Example 9

Detection of βII-spectrin and βII-SBDPs in Blood Serum of human neurotoxic insult

Human patients are screened and assessed for suffering from a neurotoxic insult. Blood serum is collected at 0, 12, 24, and 72 hours post-hypothermic therapy, centrifuged at 500 rpms and stored at −70° C. until assayed. The measurements of βII-spectrin or any of the βII-SBDPs are based on ELISA results of the participating patients. After statistical analysis of the results, modulated levels of βII-spectrin or any of the βII-SBDPs are indicative of neurotoxicity, providing an identification and risk stratification system for patients in a time frame where current diagnostic methods are unreliable. It is found that levels of βII-spectrin are found to be decreased in the injured patients than in the control patients, while levels of the βII-SBDPs, such as βII-SBDP-80, βII-SBDP-85, βII-SBDP-108, and βII-SBDP-110, are found to be at higher levels than the control samples.

### Example 10

### Detection of βII-spectrin and βII-SBDPs in Urine

Human subjects suspected of having TBI are screened and assessed. Urine is drawn at first urination after screening and again at 24, 48, and 96 hours while prepared and stored using conventional methods until assayed. The measurements of βII-spectrin or any of the βII-SBDPs are based on ELISAs and are performed blindly without knowledge of any clinical information. After statistical analysis of the results, modulated levels of βII-spectrin or any of the βII-SBDPs are indicative of detection of TBI, providing a reliable diagnostic method where standard diagnostic procedures are still silent or unreliable within the same time frame.

### Example 11

### Detection of βII-spectrin and βII-SBDPs in Saliva

Human subjects suspected of having TBI are screened and assessed. Saliva is collected by buccal swab of neonate inner cheek and tongue. Swab brushes are washed with phosphate buffered saline (PBS) and immediately stored at −80° C. until assayed. While frozen, biomarkers are extracted from the swab and prepared for assaying by ELISA. After statistical analysis of the results, modulated levels of βII-spectrin or any of the βII-SBDPs, as compared to their control counterparts are indicative of detection of HIE in the first hours following birth, providing an identification and risk stratification system for patients in a time frame where current diagnostic methods are unreliable. In addition, βII-spectrin or any of the βII-SBDPs are monitored post-treatment to gauge therapeutic response and to provide a more accurate prognosis.

### Example 12

### Detection of βII-spectrin and βII-SBDPs Using an In Vitro Diagnostic Device

Accumulation of βII-spectrin and βII-SBDPs are analyzed in the biological samples, using the processes described herein, through the use of an assay which includes antibodies raised against these peptides. These assays are then incorporated into the in vitro diagnostic devices where the methods of detection of the neurological condition are performed and the results are illustrated. The assays are for βII-spectrin and each of the βII-SBDPs outlined above, but other assays are multiplexed to include one or more combination markers. A portion of the samples are tested using on βII-spectrin and/or βII-SBDPs assays. Normal patient samples are also analyzed for the same biomarkers, and a normal metric is calculated to indicate a non-injury state. The metric is then incorporated into the in vitro diagnostic device either through a computer algorithm, or where a calorimetric indication is provided; the dyes are activated indicating injury when the level of the measured biomarker is higher than what is determined in the normal metric.

Prior to analysis, an assay is developed using a detection and capture antibody, each antibody being specific to the biomarker intended to be measured. For example, for βII-SBDP-80, a monoclonal/monoclonal pair (capture/detection) is used to detect the level of biomarkers. Notwithstanding, similar results are achieved through the use of a monoclonal/polyclonal pair, a polyclonal monoclonal pair, and a polyclonal/polyclonal pair. The assay is optimized and tested using a calibrator and spiked serum to ensure that assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample. The in vitro diagnostic devices used in this example have incorporated assays contained therein, which assays may be substituted herein using the methods therein contained.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. An in vitro diagnostic device detecting neural injury, neuronal disorder or neurotoxicity comprising:
a sample chamber for holding a first biological sample collected from a subject;
an assay module in fluid communication with said sample chamber, said assay module containing an agent for specific for detecting at least one biomarker of βII-spectrin or a breakdown product thereof wherein said assay module analyzes the first biological sample to detect the amount of the one or more biomarker present in said sample; and
a user interface, wherein said user interface relates the amount of the one or more biomarker measured in the assay module to detecting a neural injury or neuronal disorder in the subject or the severity of neural injury or neuronal disorder in the subject.
